(19) United States
(12) Patent Application Publication
(10) Pub. No.: US 2018/0228703 A1
(43) Pub. Date: Aug. 16, 2018

(54) Improved Hyaluronan and Modified-Hyaluronan in Biomedical Applications

(71) Applicant: CHL Industries, LLC, Frisco, TX (US)

(72) Inventors: John Patrick McCook, Frisco, TX (US); Peter Ladislaus Dorogi, Easton, PA (US); David Bruce Vasily, Bethlehem, PA (US)

(21) Appl. No.: 15/751,173

(22) PCT Filed: Jul. 19, 2016
(86) PCT No.: PCT/US2016/042986

§ 371 Date: Feb. 8, 2018

Related U.S. Application Data
(60) Provisional application No. 62/202,794, filed on Aug. 8, 2015.

(51) Int. Cl. A61K 8/19; A61K 31/555; A61K 31/409; A61K 31/728; A61K 8/49; A61K 8/73; A61Q 19/08; A61K 9/00

(52) U.S. Cl. CPC A61K 8/19; A61K 31/555; A61K 31/409; A61K 31/728; A61K 8/494; A61K 2800/91; A61Q 19/08; A61K 9/0019; A61K 9/0014; A61K 2800/58; A61K 8/735

(57) ABSTRACT

Disclosed are methods and compositions for reducing the rate and/or extent of degradation of endogenously-generated and/or exogenous hyaluronic acid (HA), or a modified-HA composition, by the administration, topically and/or by injection, of sodium copper isochlorin e4 or oxidized sodium copper isochlorin e4. The methods and compositions extend the shelf-life of HA or modified-HA (i.e., prior to therapeutic use) and improve therapeutic outcomes including (i) improving the appearance of the skin exhibiting rhytids, grooves, furrows, creping, sagging, or otherwise appearing hollow, (b) reducing skeletal pain in and around the knees, ankles, shoulders, elbows, wrists, distal phalanges, and spine (including facet joints and intervertebral discs), and extending the duration of such improvement in skin appearance and reduction of pain.

### FIELD OF INVENTION

The present invention is directed to methods of improving (e.g., lengthening the shelf-life of) hyaluronan (e.g., sodium hyaluronate or hyaluronic acid, or “HA”) or modified HA (e.g., HA that undergoes a crosslinking or other reaction), as well as improving the stability and reducing the rate and/or extent of degradation of HA (or modified HA) when administered to a human, in particular by injection. Additionally, the invention is directed to improving the outcomes of dermatologic, orthopedic and other surgical procedures that employ HA or modified HA. The present invention is also directed to improving (e.g., prolonging the lifespan of) biomedical devices coated with HA or modified HA.

### BACKGROUND OF THE INVENTION

Hyaluronic acid, also known as hyaluronan, and referred to in the present application by the acronym HA, is a glycosaminoglycan that consists of repeating units of two disaccharides—D-glucuronic acid and D-N-acetylglucosamine. (Typically, the carboxyl group of D-glucuronic acid is converted into its sodium salt.) Each of the disaccharide monomers has an approximate molecular weight of 400 Da and typically attaches to the other through beta-1,4 glycosidic bonds, forming an HA polymer chain. The length of HA polymer chains can reach 25,000 (or more) repeating disaccharide units, with a total molecular weight of greater than 10,000 Da.

Chemical modification of HA for therapeutic and regenerative medical application is described in the biomedical literature. See, e.g., H A Prestwich G D, Kuo J W. “Chemically-modified HA for therapy and regenerative medicine.” Curr. Pharm Biotechnol. 2008; 9:242.

Modified-HA has been divided into two groups: (i) “processed”, “fabricated” or “monolithic” HA that has been terminally modified and, therefore, cannot form new chemical bonds in the presence of cells or tissues; and (ii) “living HA” that can form new covalent bonds in the presence of cells, tissues, and therapeutic agents. (“Living” HA derivatives are typically used in 3-dimensional cell cultures, construction of tissue or tissue matrix scaffolding, and in vivo delivery of biologically active ingredients.) See, e.g., J A Burdick and G D Prestwich, “Hyaluronic Acid Hydrogels for Biomedical Applications” Adv. Mater. 2011 Mar. 25; 23(12): H41-H56. Modification of HA has been accomplished by one of the following pathways: reacting HA with adipic dihydrazide, with further crosslinking via acrylamide or hydrazone linkages; reacting HA with butane-1,4-diol diglycidyl ether; peroxidase crosslinking of HA with tyramide; formation of dialdehyde-modified HA by periodate oxidation; methacrylate modification of HA on the primary 6-hydroxyl group; esterification of HA (e.g., with benzyl ester); reaction of HA with glycidyl methacrylate; modification of carboxyl group of HA by reaction with 3,3′-di(thiopropionyl)bishydrazide (DTPH modification), followed by reduction of the disulfide bond with dithiothreitol, resulting in HA-DTPH, which, in turn, may be oxidatively crosslinked to form a hydrogel; reaction of HA with bromoacetate. A thioether crosslinked semi-synthetic ECM may be formed by crosslinking thiol-modified carboxymethyl HA (CMHA-S) with thiol-modified gelatin using polyethylene (glycol) diacrylate as the crosslinker.

Injectable compositions containing HA have been approved for medical applications, principally as dermal fillers and for use in the treatment of osteoarthritis. In aesthetic medicine (i.e., cosmetic dermatology and plastic/reconstructive surgery), injectable formulations containing HA are typically modified; more particularly, HA polymer chains are often chemically crosslinked to each other, creating a viscoelastic polymeric gel network. Two crosslinkers used in HA dermal fillers approved by the United States Food & Drug Administration are 1,4-butanediol diglycidal ether and di-vinyl sulfone. Both react with hydroxyl groups on the HA chains, and aid in slowing down enzymatic and free radical degradation of the HA polymer.

The U.S. Food and Drug Administration (FDA) has approved the use of chemically cross-linked HA gels for the following dermatological indications in patients over the age of 21: lip augmentation and dermal implantation for correction of perioral rhytids (wrinkles around the lips); deep (subcutaneous and/or supraperiosteal) injection for cheek augmentation to correct age-related volume deficit in the mid-face; injection into the mid to deep dermis for correction of moderate to severe facial wrinkles and folds (such as nasolabial folds).

In linear or uncrosslinked form, HA is known in the medical art as an excellent lubricant. This property has led to investigation of HA for increasing cushioning and providing lubrication in biological applications, and thereby providing relief from mild to moderate osteoarthritis pain in the ankle, shoulder, elbow, wrist, fingers and toes. The majority of studies to date have assessed intra-articular (IA) HA injections for knee osteoarthritis, which is the only indication currently approved by the FDA. Six preparations of intra-articular HA have been approved by the FDA as an alternative to non-steroidal anti-inflammatory drug therapy in the treatment of OA of the knee: Synvisc® and Synvisc-One®, both from Genzyme; Hyalgan®, from Fidia; Supartz®, from Smith and Nephew; OrthoVisc®, from Anika; and Euflexxa®, previously named Nuflexxa™, from Savient. Synvisc undergoes additional chemical crosslinking to create hyaluronans with increased molecular weight (6,000 kDa) compared to Hyalgan (500-730 kDa) and Supartz (620-1170 kDa). The differing molecular weights of these products results in different biopersistence; the half-life of Hyalgan® or Supartz® is estimated at 24 hours, while the half-life of Synvisc® products may range up to several days. Dosing can vary, and approved IA treatment regimens for osteoarthritic knee pain can, within the scope of the present invention, include once-weekly administration, (e.g., for three, four, or five weeks), as well as repeat administration, as needed, spaced out over more extended periods of time (e.g., once every six months).

A common starting material—non-modified (also known as “free”) HA—is typically HA in dry powder form. The powder is then mixed with water, resulting in a viscous liquid with the appearance and consistency of egg white. If such a solution were injected in tissue, enzymes such as hyaluronidase and free radicals that are present in the body would quickly degrade (i.e., cleave) non-modified HA polymers. As a result, the half-life of a free HA injectable solution in tissue would, as discussed above, be expected to be about 1-2 days. Accordingly, there has been and remains a need for improved forms of HA; for example, improved injectable forms of HA that degrade more slowly (i.e., have increased biopersistence). Such needs are met by the present invention.

In the case of dermal fillers, it is desirable to optimize the “lifting capacity” of the injected viscoelastic HA preparation. By “lifting capacity” is meant the ability to achieve a desired aesthetic correction (e.g., reduction of cutaneous rhytids, grooves and furrows, restoring volume (e.g., to sagging or “hollow” areas of the face), or contouring, by plumping or lifting the tissue and also resisting deformation.

The ability to lift tissue and resist deformation after the injection—depends on the gel strength. However, in “stronger” HA gel formulations, higher degrees of crosslinking may have an undesired effect—namely, by reducing the hydrophilicity of the HA polymer chains, the injected gel may have reduced lifting capacity. Accordingly, there remains a need for a long-acting (biopersistent) HA injectable gel that maintains or achieves optimized lifting capacity. This need is met by the compositions and methods of the present invention.

The use of cross-linked HA gels in medicine presents issues beyond biopersistence. Certain prior art modified HA gels contain unreacted cross-linkers, which have been associated with immune reactions. Moreover, the literature reports that certain prior HA gel formulations—particularly “harder” or “stiffer” gels—are not easily administered. This problem is sometimes referred to in the art as poor extrusion.

For example, clinicians have reported difficulty initiating plunger movement when injecting a stiff gel. Additionally, when an injectable HA gel is more “viscous”, clinicians often find it difficult to push the plunger at steady rate.

In response to limitations caused by viscosity and gel hardness (defined below), prior art medical uses of HA have included administering gels containing both cross-linked and non-modified (“free”) HA. The lubricating properties of low viscosity HA have been reported to lower gel hardness and the force required for injection. However, as discussed above, addition of free HA is of limited therapeutic value due to its short half-life. Accordingly, there has been and remains a need for injectable forms of free and/or modified HA that can be administered easily. This need is met by the present invention.

In certain uses, HA and its carrier can migrate into the tissues below the injection site, causing inflammation, allergic reactions and/or infections. Attempts to mitigate and avoid these potential negative sequelae have lead to the development of alternative fillers. However, these fillers also have limitations. For example, the injectable form of collagen is resorbed relatively rapidly (between 1 and 3 months). Moreover, because of its bovine or porcine origin, collagen injections are known to cause allergic reactions. There remains a need for HA injectable compositions with improved safety and efficacy. This need is met by the present invention, which in certain of its embodiments, is directed to novel compositions that a long-acting biopersistent form of HA in combination with ingredients that mitigate these negative sequelae.

The clinical literature reports that radiofrequency (RF) treatment prior to HA filler injection can provide reduction of facial lines and wrinkles (e.g., nasolabial folds) as well as correct the flattening and furrowing of the central area of the mid-cheek. The methods of the present invention improve on this prior art bi-modal therapy by providing improved, longer-lasting aesthetic improvement in terms of reducing the appearance of rhytids, grooves and furrows.

It is known in the art that copper plays an important physiological role in the health of connective tissue, notably the skin. Importantly, copper is used by norepinephrine biosynthetic enzymes and lysyl oxidase, an enzyme that plays a role in the biogenesis of connective tissue matrices by crosslinking collagen and elastin. Smith-Mungo L I, Kagan H M, “Lysyl oxidase: properties, regulation and multiple functions in biology” Matrix Biology Vol. 16, pp. 387-398 (1998); Liu X, ZhaoY, Gao J, et al. “Elastic fiber homeostasis requires lysyl oxidase-like 1 protein” Nature Genetics, Vol. 36, pp. 178-182 (2004). Cytochrome oxidases (subtypes a, b and c) are copper dependent enzymes involved in the production of ATP and generally involved in the aging process, including skin aging. By releasing copper ions, the methods and compositions of the present invention provide improved longer-lasting aesthetic improvement in terms of reducing the appearance of rhytids, grooves and furrows and also reduce post-injection site bruising.

The viscosity of HA solutions is known to decrease over time, especially when stored above room temperature. This viscosity drop is even more pronounced when storage temperatures exceed 60° C. See, Lowry, Karen M. and Beavers, Ellington M.; “Thermal Stability of Sodium Hyaluronate in Aqueous Solution” J. Biol. Mat. Res.; Vol. 28, pp. 1239-1244 (1994).

Additionally, it is well known in the biomedical arts that HA is susceptible to chemical degradation (e.g., by oxidation and/or hyaluronidase). See, L. Sŏltés et al. “Degradation of High-Molar-Mass Hyaluronan and Characterization of Fragments” Biomacromolecules, Vol. 8, No. 9, pp. 2697-2705 (2007)(Oxidative degradation not only reduces the molecular size of hyaluronan but also modifies its component monosaccharides, generating polymer fragments that may have properties substantially different from those of the original macromolecule.)

The methods and compositions of the present invention address the above described shortcomings—adding a Chlorophyllin Compound, or Chlorophyllin Copper Complex Sodium (each as defined below) to a composition that contains HA (or modified HA) increases the long-term stability of the composition, not only by retarding thermal degradation and decreased viscosity but also retarding chemical and biological degradation (e.g., by oxidation and/or hyaluronidase).

### SUMMARY OF THE INVENTION

A first aspect of the present invention is directed to methods and compositions for reducing the rate and/or extent of degradation of endogenously-generated HA by the administration of CHLcu or a CHL compound, either topically or by injection.

A second aspect of the present invention is directed to methods and compositions for reducing the rate and/or extent of degradation of non-native i.e., (exogenous) HA (or a modified HA compound) by co-administration of CHLcu or a CHL compound, either topically or by injection.

A third aspect of the present invention is directed to methods and compositions for extending the shelf-life of HA or modified-HA prior to use (i.e., for biomedical or therapeutic purposes) with CHLcu or a CHL Compound.

A fourth aspect of the present invention is directed to methods and compositions for improving therapeutic outcomes when CHLcu or a CHL Compound plus HA (or modified-HA) is administered by injection or topically, or a biomedical device that is coated or constructed with CHLcu or a CHL Compound plus HA (or modified HA) and is inserted (temporarily or permanently) in the human body. Non-limiting examples of improved therapeutic outcomes achieved by practicing the methods of the present invention include (a) improving the appearance of the skin exhibiting rhytids, grooves, furrows, creping, sagging, or otherwise appearing hollow, (b) reducing skeletal pain in and around the knees, ankles, shoulders, elbows, wrists, distal phalanges, and spine (including facet joints and intervertebral discs). Improved therapeutic outcomes are also seen in terms of extending the duration of improvement in the appearance of human skin and reduction of skeletal pain.

A fifth aspect of the invention includes the use of CHLcu or CHL Compounds, applied topically or by injection, to upregulate and thereby increase gene expression of human extracellular matrix proteins and key biological building blocks of human epidermal and dermal tissue including procollagen-1, and fibrillin-1.

The above described methods and compositions are accomplished introducing CHLcu or one or more of CHL Compounds selected from the group consisting of sodium copper chlorophyllin complex, sodium isochlorin e4, oxidized sodium isochlorin e4, sodium copper isochlorin e4, oxidized sodium copper isochlorin e4, sodium magnesium isochlorin e4, and/or oxidized sodium magnesium isochlorin e4 into a micro-environment (e.g. body part that has or is undergoing a therapeutic procedure) containing HA (or modified-HA) or a formulation containing HA (or modified-HA) or capable of stimulating the production of endogenous HA.

### DETAILED DESCRIPTION OF THE INVENTION

As used in the present invention, “CHL Compound” is to be understood to mean CHLCu (defined below), sodium isochlorin e4, oxidized sodium isochlorin e4, sodium copper isochlorin e4, oxidized sodium copper isochlorin e4, sodium magnesium isochlorin e4, and/or oxidized sodium magnesium isochlorin e4, as well as water-soluble alkali salts of the above compounds (e.g., potassium, lithium.)

As used in the present invention, “Chlorophyllin Copper Complex Sodium” also known as sodium copper chlorophyllin complex, copper chlorophyllin complex, and copper chlorophyllin (abbreviated “CHLcu”) means a chemical complex of water soluble copper porphyrin compounds containing one or more of sodium isochlorin e4, oxidized sodium isochlorin e4, sodium copper isochlorin e4, and/or oxidized sodium copper isochlorin e4. The composition of CHLCu is further defined in the US Pharmacopoeia (“USP”), 39th Edition, the disclosure of which is incorporated herein by reference. United States Pharmacopiea 39th Edition. USP is published by The U.S. Pharmacopeial Convention, a scientific nonprofit organization that sets standards for the identity, strength, quality, and purity of medicines, food ingredients, and dietary supplements manufactured, distributed and consumed worldwide. USP's drug standards are enforceable in the United States by the Food and Drug Administration, and these standards are used in more than 140 countries. Monographs for drug substances, dosage forms, and compounded preparations are featured in the USP.

The aforementioned magnesium isochlorin compounds can be found in Sodium Chlorophyllin Magnesium Complex available from Chlorophyll MM from Food Ingredient Solutions, LLC (Teterboro, N.J.).

As used in the present invention, “hyaluronic acid” (abbreviated “HA”) is a biocompatible, non-immunogenic, linear polysaccharide made of repeating disaccharide units of d-glucuronic acid and N-acetyl glucosamine linked by glucosidic bonds. HA may be isolated from mammals (e.g., bovine vitreous humor or rooster combs), produced in genetically modified microorganisms, or synthesized (e.g., De Luca et al., J. Am. Chem. Soc., 1995: 117; 21, pp. 5869-5870.)

Preferably, in methods of the present invention in which hyaluronic acid (or its derivative) is injected, HA is derived from Streptococcus equi.

As used in the present invention, modified-HA is to be understood to mean HA that is modified, for example, by cross-linking, conjugation, inverting one or more stereocenters and/or modifying, replacing or removing one or more substituents on the disaccharide backbone. Common modifications include chemical reactions occurring at (i) the glucuronic acid carboxylic acid of HA, (ii) the primary or secondary hydroxyl group of HA, or (iii) the N-acetyl group (after deamidation) of HA.

Modified HA compounds may be created by an esterification reaction in which an alcohol is reacted with the carboxylic acid group of the glucuronic acid subunit of HA or a carboxylic acid is reacted with a hydroxyl moiety on disaccharide backbone.

Modification may also be achieved by etherification in which the hydroxyl group of the disaccharide backbone is reacted under basic conditions with, for example, glycidyl methacrylate or methacrylic anhydride.

HA may also be modified by amidation or coupling disulfide-containing reagents to the carboxylic acid group of the disaccharide backbone by reduction to expose the thiol groups.

In certain embodiments in which the methods of the present invention involve administration of modified-HA, the carboxylic acid group on HA is modified by reaction with one of the following compounds: a methacrylate; a hydrazide; an amine; an organo-sulfur compound; or an aldehyde.

Methacrylates suitable for forming modified-HA compounds useful in practicing the methods of the present invention include 2-aminoethyl methacrylate hydrochloride (AEMA) and 2-aminoethyl methacrylate (APMA).

Hydrazides suitable for forming modified-HA compounds useful in practicing the methods of the present invention include adipic acid dihydrazide (ADH) and carbodihydrazide.

Amines suitable for forming modified-HA compounds useful in practicing the methods of the present invention include hexane-1,6-diamine (HDMA) and pyridyldithioethylamine (PDPH).

Acrylate-modified HA suitable for use in practicing the methods of the present invention may be formed in dimethyl sulfoxide by mixing HA (tetrabutylammonium (TBA) salt, (benzotriazol-1-yloxy)tris(dimethylamino) phosphonium hexafluorophosphate, AEMA and N,N-diisopropylethylamine.

Methacrylate-modified HA suitable for use in practicing the methods of the present invention may be formed by using two mole equivalent 1-ethyl-3-(3-dimethyl-aminopropyl)carbodiimide (EDC) and N-(3-aminopropyl)methacrylamide or using glycidyl methacrylate with TBA bromide as a catalyst.

Organo-sulfur compounds that may be used to create modified HA compounds suitable for use in practicing the methods of the present invention include thiols and vinyl sulfonyls (VS).

Modified-HA compounds suitable for use in practicing the methods of the present invention may be formed by a carbodiimide coupling reaction in which an aqueous solution of HA is combined with EDC at a pH of about 4.75.

Carbodiimide coupling reactions that may be used to create modified-HA compounds may be performed in the presence of a nucleophilic catalyst. Some carbodiimide coupling reactions employ a nucleophilic catalyst having a pKa of between about 2 and about 3.5. Non-limiting examples of nucleophiles with such a pKa are hydrazides and aminooxy derivatives. Other carbodiimide coupling reactions may also be performed at a higher pH—for example, at a pH of from about 6 to about 7, preferably in the presence of a nucleophilic catalyst such as N-hydroxybenzotriazole or N-hydroxysuccinimide.

One preferred thiol-modified HA suitable for use in practicing the methods of the present invention may be prepared by conjugating a disulfide-containing molecule using EDC chemistry. Non-limiting examples of disulfide-containing molecules include cystamine and 3,3′-dithiopropionic acid dihydrazide. In these reactions, the disulfide bond may be reduced with dithiothreitol.

In further embodiments, HA may be modified by selective deprotection of the acetyl group of N-acetyl-glucosamine, followed by a reaction at the amino residue. Aldehyde-modified HA may be prepared via partial oxidation of the HA sugar backbone using sodium periodate. In one embodiment, aldehyde-modified HA may be prepared by grafting amino glycerol units onto HA via EDC chemistry, by selectively oxidization of the vicinal diols of glycerol with periodate.

Other methods of preparing aldehyde-modified HA include use of radical-based oxidants, such as 2,2,6,6-tetramethyl-1-piperidinyloxy, and radical generation of double bonds in the glucuronic acid component of HA with hyaluronate lyase followed by ozonolysis and reduction.

Amphiphilic HA derivatives may be formed by alkylation of the carboxylic acid groups on HA using HA-TBA salt and an alkyl halide (e.g., bromide or iodide) in an organic solution. Alkylated HA can also be formed by reaction HA modified with adipic acid and 1-decanal in an aqueous medium.

The hydroxyl group in HA may be modified by etherification, divinylsulfone (DVS) crosslinking, esterification and bis-epoxide crosslinking.

Epoxides may also be used as a crosslinker to form HA hydrogels. One particularly preferred crosslinking agent is butanediol diglycidyl ether (BDDE). Crosslinking of HA with BDDE is performed in a 0.25 M solution of sodium hydroxide. In this crosslinking reaction, the epoxide ring opens to form ether bonds with the hydroyxyl groups of HA.

Reacting HA with DVS at high pH values (pH greater than about 13) creates sulfonyl bis-ethyl cross-linkages between the hydroxyl groups of HA.

Additional methods for chemically modifying HA, as well as synthetic routes for obtaining HA derivatives, are well known to the skilled artisan and are described below as well as in C. E. Schanté et al. Carbohydrate Polymers, Vol. 85, pp. 469-489 (2011), the disclosure of which is incorporated by reference in its entirety.

As used in the present invention, modified HA compound is also to be understood to include “small molecule” drugs, as well as peptides, proteins and biological drugs, polymers and other molecules containing primary amine groups that are conjugated to HA via the above-mentioned EDC chemistries.

In certain preferred embodiments of the present invention that employ a modified form of hyaluronic acid, the HA is crosslinked HA by reacting “free” HA with a crosslinking agent under suitable reaction conditions. Non-limiting examples of crosslinking agents that can be used to form modified (crosslinked) hyaluronic acid suitable for use in performing the methods of the present invention include 1,4-butanediol diglycidyl ether, di-vinyl sulfone, 1,4-bis(2,3-epoxypropoxy)butane, 1,4-bisglycidyloxybutane, 1,2-bis(2,3-epoxypropoxy) ethylene, 1-(2,3-epoxypropyl)-2,3-epoxycyclohexane, pentaerythritol tetraglicidyl, and sodium glucuronate-N-acetylglucosamine.

In certain particularly preferred embodiments, sodium hyaluronate is cross-linked with 1,4-butanediol-diglycidyl ether or di-vinyl sulfone.

In other preferred embodiments, the compositions used in performing the methods of the present invention are comprised of a mixed cross-linked gel of hyaluronic acid, or a derivative thereof, preferably, sodium hyaluronate, and at least one other hydrophilic polymer having a functional group capable of reacting with divinyl sulfone.

In non-limiting examples of this preferred embodiment, the mixed cross-linked gel is comprised of sodium hyaluronate having a molecular weight of about 50,000 to about 8 million Da and the hydrophilic polymer having a functional group capable of reacting with divinyl sulfone is hydroxyethyl cellulose, carboxymethyl cellulose, xanthan gum, chondroitin sulfate, heparin, collagen, elastin, albumin, keratin sulfate, or a sulfated aminoglycosaminolgycan.

In the preferred embodiment described in the immediately preceding paragraphs, hyaluronic acid (or its derivative), preferably sodium hyaluronate, is mixed with one of the aforementioned hydrophilic polymers having a functional group capable of reacting with divinyl sulfone. The mixing process may be performed in the presence of di-vinyl sulfone in a dilute aqueous alkaline solution, preferably a pH of not less than about 9, preferably at a temperature of about 20° C. In particularly preferred embodiment, the ratio of the sodium hyaluronate to divinyl sulfone is from 15:1 to 1:5 by weight.

Injectable compositions containing hyaluronic acid—non-modified (“free”) HA, modified HA, or a combination of “free” HA and modified-HA—may, and in certain preferred embodiments, do contain one or more polymeric hydrogels, non-limiting examples of which include chitosan, alginates, gelatin, pectin, sodium carboxy-methylcellulose, polyvinyl alcohol, polyvinylpyrrolidone, Poly-L-Lysine, Polyamidoamine dendrimers, Polyethyleneimines, Poly-lacto-co-glycolic acid, Polyethylene glycol-PLGA-Polyethylene glycol hydrogels, Polylactic and Polyglycolic hydrogels.

The inventive compositions of the present invention—namely, a combination of HA (and/or modified HA) and CHLcu or a CHL compound—may be administered topically and/or by injection, either together, or separately, in a variety of forms, including solutions, suspensions, emulsions, liposomal dispersion, films, gels, foams and scaffolds.

Additionally, the inventive compositions of the present invention—combinations of HA (and/or modified HA) and CHLcu or a CHL compound—may be used to coat a biomedical device, non-limiting examples of which include catheters, stents, and scaffolding.

A first aspect of the present invention is directed to methods and compositions for reducing the rate and/or extent of degradation of endogenously-generated HA by the administration of CHLcu or a CHL Compound, either topically or by injection.

A second aspect of the present invention is directed to methods and compositions for reducing the rate and/or extent of degradation of non-native i.e., (exogenous) HA (or a modified HA compound) by co-administration of CHLcu or a CHL Compound, either topically or by injection.

Related to the first and second aspects of the present invention are methods and compositions for extending the biological residence time of HA (or a modified HA compound) and/or improving the physicochemical integrity and stability of an HA compound (or modified HA compound), including mechanical properties such as elasticity, modulus of elasticity, and lubricity by introducing (i.e., administering) CHLcu or a CHL Compound into a micro-environment (e.g. body part that has or is undergoing a therapeutic procedure) containing endogenous HA or a formulation containing HA (or modified HA).

As used in the present application, “improvement” in the physicochemical integrity and stability of an HA compound (or a modified HA compound) is to be understood to include, but not be limited to, one or more of the following properties: reduced thermal degradation, as measured by a change in viscosity over time (for example, 8 weeks) at an elevated temperature simulating body temperature, for example, 40° C.; and reduced chemical and/or biological degradation (e.g., by hyaluronidase and/or oxidation).

Products containing HA and/or a modified HA compound (solutions, suspensions, emulsions, liposomal dispersion, films, gels, foams and scaffolds) show decreases in viscosity of about 25% when stored at 25° C. for several months (e. g, 8 weeks). However, when stored at 40° C. for a similar period of time (e.g., 8 weeks), the viscosity of a product containing HA or modified HA viscosity decreases greater than 25%, and loses more than 50% of initial viscosity when stored at 50° C. for 8 weeks.

Surprisingly and unexpectedly, when a product (injectable or topical composition) that is comprised of HA (or modified HA) is stored in the presence of CHLcu, at a concentration of from about 50 to about 250 ppm at a temperature of 40° C. for about 8 weeks, the viscosity of the preparation was observed to increase by up to 25% and stabilize to within 5% of the baseline viscosity after 8 weeks of storage.

In addition, Applicants have surprisingly and unexpectedly discovered that products containing HA and/or a modified HA compound are not degraded as rapidly when exposed to hyaluronidase. This reduced rate of degradation (i.e., increased stability or integrity) is manifested in reduced loss of viscosity when a product containing HA (or modified HA) and CHLCu is exposed (i.e., challenged, with hyaluronidase).

A third aspect of the present invention is directed to methods and compositions for extending the shelf-life of HA or modified-HA prior to use (i.e., for biomedical or therapeutic purposes). Extended shelf-life (as a result of being stored in an environment that contains CHLcu or a CHL Compound) may be measured, for example, by changes in viscosity using the previously described test methods (i.e., over a defined temperature range and/or when stored in the presence of various concentrations of hyaluronidase for extended periods of time). Extended shelf-life may be measured, for example, by changes in the amount or type of degradant(s).

A fourth aspect of the present invention is directed to methods for improving the appearance of human skin exhibiting rhytids, grooves, furrows, creping, sagging, or otherwise appearing hollow by topical administration and/or injection of CHLcu or one or more of CHL Compounds alone or in combination with HA and/or modified-HA.

Methods in accordance with this fourth aspect of the invention improve appearance of human skin in terms of one or more aesthetic parameters selected from: (i) reducing the number, length and/or depth of rhytids, grooves and furrows, especially of the face (ii) adding, restoring volume (e.g., plumping) or otherwise defining/contouring areas of skin that exhibit creping, sagging, or otherwise appear “hollow”.

The improvement in the appearance of human skin can be measured using digital clinical photography and computer image analysis that measures depth, width, and/or length of wrinkles/fine lines and assigns a numerical score. One such computer analysis system is VISTA®-CR system from Canfield Imaging Systems (Parsippany, N.J.). Changes (i.e., reduction) in the number/depth of wrinkles/fine lines may also be assessed visually by a trained observer.

In certain embodiments, the area in need of aesthetic improvement (i.e., improved appearance) is injected one or more times with a preparation comprised of (a) CHLcu or one or more of sodium isochlorin e4, oxidized sodium isochlorin e4, sodium copper isochlorin e4, oxidized sodium copper isochlorin e4, sodium magnesium isochlorin e4, and/or oxidized sodium magnesium isochlorin e4 and (ii) hyaluronic acid, or a derivative thereof, preferably, sodium hyaluronate.

In other embodiments, a topical preparation comprised of CHLcu or one or more CHL Compounds is applied to the area in need of aesthetic improvement.

In these embodiments, the area in need of aesthetic improvement can also be injected with hyaluronic acid, or a derivative thereof, preferably, sodium hyaluronate, or a modified HA compound.

Type I collagen—coded by the gene COL1A1—is the most abundant proteinaceous ingredient in the skin and is responsible for many of the skin's key physico-chemical properties. Aging is characterized by reduction in the amount of Type I collagen, and an increase in its degradation and glycation.

COL15A1 is the gene that codes for a fibril-associated collagen important for tensile strength of the skin; localized in the dermal-epidermal junctions, it important for skin, muscle and micro-vessel integrity.

Type V collagen—coded by the COL5A1 gene—is found in tissues containing type I collagen and is involved in regulating the assembly of heterotypic fibers composed of both type I and type V collagen.

A fifth application of the present invention is therefore directed to methods for increasing the expression of one or more genes that code for extracellular matrix proteins, including, procollagen-1 and fibrillin-1, by topical application of CHLcu or a CHL Compound.

A sixth aspect of the present invention is directed to methods for reduction of skeletal pain, especially in and around the knees, ankles, shoulders, elbows, wrists, distal phalanges, and spine (including facet joints and intervertebral discs) by topical administration and/or injection of CHLcu or one or more CHL Compounds alone, and/or in combination with HA or modified-HA.

In certain embodiments, the area in need of pain relief/reduction is injected one or more times with a preparation comprised of (a) CHLcu or one or more CHL Compound(s) and (ii) hyaluronic acid, or a derivative thereof, preferably, sodium hyaluronate, and/or in combination with one (or more) modified HA compounds.

In other embodiments, a topical preparation comprised of CHLcu or one or more CHL Compound(s) is applied to the area in need of pain relief/reduction. In these embodiments, the area in need of pain relief/reduction may also be injected with hyaluronic acid, or a derivative thereof, preferably, sodium hyaluronate, and/or in combination with one (or more) modified HA compounds.

In one embodiment of the present invention, the at least one HA and/or modified-HA compound is/are present in the finished composition (injectable or topical preparation) at a molecular weight ranging from at least about 10,000 Da to about 3.0 million Da.

In certain embodiments of the methods of the present invention, the CHLcu or one or more CHL Compound(s) is/are present in the finished composition (injectable or topical preparation) at a concentration of between 1 and 1000 mcg/mL on a weight/weight based on the total weight of total composition.

In certain preferred embodiments, in which the methods of the present invention are practiced by topical administration of HA (or modified HA) in combination with CHLcu (or one or more CHL Compounds), the CHLcu or one or more CHL Compound(s) is administered at a concentration of from about 100 to about 1,000 mcg/mL, even more preferably from about 250 to about 1,000 mcg/mL.

In certain preferred embodiments, in which the methods of the present invention are practiced by injecting HA (or modified HA) and CHLcu (or one or more CHL Compounds), the CHLcu or one or more CHL Compound(s) is administered at a concentration of from about 10 to about 500 mcg/mL, even more preferably from about 50 to about 250 mcg/mL.

In other embodiments, the compositions of the present invention (topical and injectable), and methods of using the same, contain CHLcu or at least one CHL Compound at a concentration of from 5 ppm to 1000 ppm (0.0005-0.1%, based on the total weight of the composition), and preferably have an isochlorin e4 content of at least 10 ppm, based on the total weight of the composition. In certain particularly preferred embodiments, the compositions (topical and injectable) used in practicing the methods of the present invention contain an isochlorin e4 at a concentration of at least 25 ppm.

In certain preferred embodiments, methods of the present invention (employing either or both a topical composition and an injectable composition) comprise the step of administering CHLcu or a CHL Compound having at least about 4% by weight that is chelated copper, and no more than about 0.25% by weight that is ionic copper.

In injectable preparations used in practicing the methods of the present invention, the preferred dry weight concentration of the hyaluronic acid (or HA derivative) or modified HA is between 18-28 mg per milliliter of “finished” injectable composition.

In certain embodiments of the present invention, non-modified (i.e., free) hyaluronic acid can be administered in an injectable composition.

In other embodiments of the present invention, cross-linked hyaluronic acid can be administered in an injectable composition.

In still other embodiments of the present invention, both non-modified (free) hyaluronic acid and cross-linked hyaluronic acid can be administered in an injectable composition. In certain of these embodiments, 10-20% of the hyaluronic acid (or its derivative) is crosslinked.

As used in the present application, the degree of crosslinking indicates the percentage of HA disaccharide monomer units that are bound to a cross-linker molecule. For example, a dermal filler within the scope of the present invention may be described as having a degree of crosslinking of 4%, by which the skilled artisan would understand that that such a filler would have, on average, four crosslinker molecules for every 100 disaccharide monomeric units of HA. All cross-linker molecules linked to HA, whether they create a cross-link or not, are included in calculating degree of crosslinking.

In embodiments of the present invention in which injectable compositions containing cross-linked hyaluronic acid are administered, the cross-linked HA preferably has a degree of crosslinking of less than about 5%.

In other embodiments in which injectable compositions containing cross-linked hyaluronic acid are administered, the injectable composition contains cross-linked hyaluronic acid having a degree of crosslinking of less than 2%.

Some injectable formulations used in practicing the methods of the present invention may be gels.

HA injectable compositions of varying gel hardness can be used within the scope of the present invention. As used in the present invention, the term “gel hardness” refers to the stiffness of the HA gel formulation, namely its resistance to being deformed elastically (non-permanently) when a force is applied to the gel. This property is often expressed as elastic modulus (G′) defined by the equation G′=stress/strain. Preferably, modified HA gels suitable for use in the compositions and methods of the present invention have a G′ modulus of from about 100 Pa to about 700 Pa.

Firm gels are not easily deformed and should therefore preferably be sized with a narrow (i.e., tight) particle size distribution range such that the particles can easily pass through a higher gauge needle (preferably 27-30). Softer gels can be more easily deformed to pass through the needle and can therefore have a broader particle size distribution than firmer gels. Irrespective of gel hardness, gels useful in the present invention preferably have an average particle size of from about 300 microns to about 700 microns. Particle size and distribution measurements are determined using particle analyzers known in the art, including from Malvern Instruments LTD.

Injectable HA gels can, and preferably do, contain a total HA concentration (amount of HA per milliliter of finished product) below the equilibrium hydration level of HA, thereby allowing the formulation to swell after injection (i.e., by taking up water from surrounding tissue).

In certain embodiments, total HA concentration in an injectable gel formulation is from about 15 to about 30 mg of HA per ml of water.

In certain embodiments, injectable formulations used in practicing the methods of the present invention are solutions comprised of HA having a molecular weight greater than about 500,000 Da.

In preferred embodiments, the CHLcu or CHL Compound is present in a hyaluronic acid injectable composition as a stabilizing agent at a concentration of from 50 ppm to 250 ppm (0.005-0.025% wt/wt).

One chemical raw material concentrate that contains sodium salts of isochlorin e4 and is suitable for inclusion in the compositions used in practicing the methods of the present invention is sold under the tradename Phytochromatic MD®, a registered trademark of CHL Industries (Frisco, Tex.).

Compositions used in practicing the methods of the present invention provide superiority over the prior art in at least the following respects: quicker, more noticeable reduction in visible signs of aging; extended duration of plumping/filling of rhythids, grooves and furrows; longer-lasting pain relief in joint pain. Without wishing to be bound by a theory, Applicant believes that these properties are attributable not only to thermal stabilization of HA viscosity and inhibition of hyaluronidase activity but also to increasing the expression of genes that code ECM proteins. More particularly, Applicants believe that the methods and compositions stimulate the production of collagen, fibronectin, and reduce degradation of glycosaminoglycans (GAGs), and that new collagen and elastin serves to stabilize (i.e., localize in place) dermal filler injections.

In intra-articular injections, Applicants believe that the methods and compositions of the present invention help stimulate the production of new cartilaginous tissue. Additionally, Applicants believe that the methods and compositions of the present invention upregulate norepinephrine levels, causing vasoconstriction of the microvasculature at the injection site, and thereby reduce post-injection site bruising.

Methods of the present invention can also include the further step of administering radiofrequency treatment, microneedle procedures and or iontophoresis or electrophoresis at the time of topically applying or injecting a preparation containing CHL or CHL Compounds with or without hyaluronic acid (or its derivative) or modified HA.

Non-limiting examples of therapeutic applications of compositions used to practice the methods of the present invention include the following:

Improved therapeutic outcomes can be achieved during “reconstruction” of various parts of the body, including skin, the musculoskeletal system, cranio-maxillofacial structures, extremities, breasts, trunk and external genitalia, by injecting compositions of the present invention.

In the context of aesthetic facial surgery (also known as plastic, cosmetic or reconstructive surgery), compositions of the present invention may be injected for soft tissue augmentation. Such compositions, also known in the art as injectable implants, dermal fillers, or wrinkle fillers, may be injected into one or more of the periorbital, malar, forehead, temporal, glabellar, mandibular and/or perioral zones.

Injections with compositions of the present invention may be used in aesthetic surgical or dermatologic procedures to create a more youthful appearance of the body or skin after removal of excess/sagging skin of the abdomen, inner thighs, and/or upper arms, (e.g., after pregnancy or significant weight loss). Cosmetic dermatologists and plastic surgeons may also administer compositions of the present invention to reduce enlarged skin pores.

Administration of compositions of the present invention may be used before or after surgical procedures to promote wound healing, including by promoting tissue neovascularization, neocollagenesis, neoangiogenesis, as well as creating a microenvironment that promotes migration/recruitment of fibroblasts, endothelial cells and keratinocytes, and/or increasing levels of growth factors. One non-limiting example of such a therapeutic application is administering a composition of the present invention before or after a chemical peel or laser skin resurfacing procedure.

Compositions of the present invention may be incorporated in wound grafts to treat chronic wounds in patients with impaired healing (e.g., diabetes).

Topical application and/or injection of compositions of the present invention may be used to improve one or more of skin elasticity, plasticity, level of skin hydration.

Skin hydration may be measured based on changes in dielectric constant due to skin surface hydration using methods known in the art, including a Corneometer®, a hand-held probe from Courage+Khazaka Electronics GmbH. Skin hydration may also be measured in terms of water evaporation from the skin, a biophysical property known in the art as transepidermal water loss (TEWL). A Tewameter®, a hand-held probe from Courage+Khazaka Electronics GmbH, may be used to measure. The rate of evaporation—change in water transported (dm) over time (dt)—is calculated according to the following formula:

$$\frac{dm}{dt} = - D \cdot A \cdot \frac{dp}{dx}$$

where A=surface [m²]; m=water transported [grams]; t=time [hours]; D=diffusion constant [=0.0877 g/m(h(mm Hg))]; p=vapour pressure of the atmosphere [mm Hg]; x=distance from skin surface to point of measurement [meters].

In performing orthopedic procedures (e.g., repair of cartilage or bone), surgeons may administer (e.g., inject) a composition of the present invention or implant a biomedical device that contains or is coated with HA (modified HA) and CHLcu or a CHL Compound. Such compositions or devices may further comprise mesenchymal stem cells, chondrogenic cells, an angiopoietin, or a growth factor.

In a broader, related application, compositions of the present invention may be used to provide controlled or sustained delivery of peptides, proteins and other “active” pharmacological or biological agents. In these “controlled release” applications, the rate of delivery of the active ingredient, as well as other aspects of pharmacokinetics (t_{1/2}, Cₘₐₓ, AUC₀₋₂₄, etc.) is controlled by molecular weight of the HA or modified HA and the amount of CHLcu or CHL Compound, which serves to stabilize the viscosity of the composition by retarding the thermal and/or chemical degradation of HA (or modified HA).

Methods of the present invention—administration of CHLcu or a CHL Compound and HA (and/or modified HA)—may be used for prevention of intraperitoneal and pericardial adhesions after intra-abdominal, pelvic and gynecologic, and cardiac surgery.

Compositions of the present invention (CHL Compound in combination with HA and/or modified HA) may be mixed with gelatin or other natural polymers and living human cells to create a 3-D “bioprinting” media to construct “living macrotissues”.

Compositions of the present invention (CHLcu or CHL Compound in combination with HA or modified HA) may be used in interventional cardiology. Administration of compositions of the present invention—in particular in the form of implanted, eluting biomedical device (i.e., a stent)—can be used in the treatment or prevention of atherosclerosis, ischemic cardiomyopathy, and atherothrombic stroke. Additionally, compositions of the present invention may be combined with valvular interstitial cells to construct heart valve tissue matrix for heart valve repair.

Topical compositions suitable for use in accordance with the methods of the present invention are illustrated in Example 1 below. In this Example, three formulations are provided in which CHLcu or one or more of sodium isochlorin e4, oxidized sodium isochlorin e4, sodium copper isochlorin e4, oxidized sodium copper isochlorin e4, sodium magnesium isochlorin e4, and/or oxidized sodium magnesium isochlorin e4 are present at the indicated concentration—0.01%, 0.025% or 0.05%—and individually, or collectively, is/are referred to as a Chlorin Composition.

Examples 2 and 3 describe clinical studies in which a cosmetic dermatologist or plastic surgeon injects a hydrogel comprised of cross-linked sodium hyaluronate and free hyaluronic acid into the mid-deep dermis of areas of a patient's skin exhibiting deep lines and folds. A topical composition of Example 1 is self-administered by the patient twice daily to the injected areas. In Example 3, the hydrogel is comprised of (i) a mixture of cross-linked sodium hyaluronate and free hyaluronic acid and (ii) CHLcu or one or more of sodium isochlorin e4, oxidized sodium isochlorin e4, sodium copper isochlorin e4, oxidized sodium copper isochlorin e4, sodium magnesium isochlorin e4, and/or oxidized sodium magnesium isochlorin e4.

Examples 4 and 5 describe clinical studies in which a rheumatologist or orthopaedic surgeon administers a hydrogel comprised of cross-linked sodium hyaluronate and free hyaluronic acid into an osteoarthritic knee. A topical composition of Example 1 is self-administered by the patient twice daily to the injected area. In Example 5, the hydrogel is comprised of (i) a mixture of cross-linked sodium hyaluronate and free hyaluronic acid and (ii) CHLcu or one or more of sodium isochlorin e4, oxidized sodium isochlorin e4, sodium copper isochlorin e4, oxidized sodium copper isochlorin e4, sodium magnesium isochlorin e4, and/or oxidized sodium magnesium isochlorin e4.

### Example 1—Topical CHL Compositions

| | CHLcu or Isochlorin Composition | | |
|---|---|---|---|
| Ingredient Name | 0.01% | 0.025% | 0.05% |
| Purified Water | 86.7289 | 86.7139 | 86.6889 |
| Chlorin Composition | 0.01 | 0.025 | 0.05 |
| Carbomer 980 | 1.1 | 1.1 | 1.1 |
| 1,3-Butylene Glycol | 3.91 | 3.91 | 3.91 |
| Sodium Lactate, 60% | 1.6 | 1.6 | 1.6 |
| Pentylene Glycol | 4.0 | 4.0 | 4.0 |
| Phenoxyethanol | 1.026 | 1.026 | 1.026 |
| Sodium Hydroxide, 33% | 1.59 | 1.59 | 1.59 |
| Vitamin E Acetate | 0.01 | 0.01 | 0.01 |
| Sodium Ascorbate | 0.005 | 0.005 | 0.005 |
| 30% Simethicone Emulsion | 0.0001 | 0.0001 | 0.0001 |
| Lecithin 90G or 90H | 0.02 | 0.02 | 0.02 |

Using a mixer, Carbomer 980 is slowly added and mixed with 90% of the water. The Carbomer/Water mixture is heated to 55° C. until the Carbomer is uniformly dispersed. 5% of the water is mixed with the CHLcu or Isochlorin Composition; the resulting Isochlorin Solution is set aside. 5% of the water is mixed with sodium hydroxide; the resulting solution is set aside. Under continual mixing, each of the remaining ingredients is added to the Carbomer/Water and mixed until uniform. Next, the sodium hydroxide solution is slowly added until fully dispersed and a uniform gel forms. Finally, the Isochlorin Solution is added and mixed.

### Examples 2-3

40 women between the ages of 35 and 50 exhibiting deep facial lines and folds are recruited to participate in a six-month study. Prior to the study, baseline digital clinical photographs are taken of each participant. Areas of the face of each patient exhibiting deep facial lines and folds are injected with a mixture of cross-linked sodium hyaluronate and free hyaluronic acid. Participants assigned to a first group, Group A, comprised of ten patients are provided with a topical composition according to Example 1, which they are instructed to administer at the injection sites twice daily—morning and night. A second group, Group B, comprised of ten patients are provided with a “control” composition identical to the composition provided to Group A, except that that the control composition does not contain an isochlorin composition. Group B participants are instructed to administer the provided composition at the injection sites twice daily—morning and night. At intervals of 2, 4 and 6 months, digital clinical photographs are again taken of each participant. The deep lines and folds in Group A appear less pronounced (i.e., remain filled/plumped) than Group B.

A third group, Group C, is injected with a hydrogel comprised of (i) a mixture of cross-linked sodium hyaluronate and free hyaluronic acid and (ii) CHLcu or one or more of sodium isochlorin e4 oxidized sodium isochlorin e4, sodium copper isochlorin e4, oxidized sodium copper isochlorin e4, sodium magnesium isochlorin e4, and/or oxidized sodium magnesium isochlorin e4. A fourth group, Group D, is injected with a hydrogel comprised of a mixture of cross-linked sodium hyaluronate and free hyaluronic acid, but without CHL or an isochlorin composition. At intervals of 2, 4 and 6 months, digital clinical photographs are again taken of each participant. The deep lines and folds in Group C appear less pronounced (i.e., remain filled/plumped) than Group D.

### Examples 4-5

40 men and women between the ages of 45 and 60 diagnosed with osteoarthritis of the knees are recruited to participate in a six-month study. Ten participants are assigned to a first group, Group E, and are provided with a topical composition according to Example 1, which they are instructed to administer at the injection sites twice daily—morning and night. Ten participants in a second group, Group F, are provided with a “control” composition identical to the composition provided to Group E, except that that the control composition does not contain an isochlorin composition. Group F participants are instructed to administer the provided composition at the injection sites twice daily—morning and night. At intervals of 2, 4 and 6 months, participants are asked to report level of pain relief. Participants in Group E report greater pain relief than Group F.

A third group, Group G, is injected with a hydrogel comprised of (i) a mixture of cross-linked sodium hyaluronate and free hyaluronic acid and (ii) CHLcu or one or more of sodium isochlorin e4, oxidized sodium isochlorin e4, sodium copper isochlorin e4, oxidized sodium copper isochlorin e4, sodium magnesium isochlorin e4, and/or oxidized sodium magnesium isochlorin e4. A fourth group, Group H, is injected with a hydrogel comprised of a mixture of cross-linked sodium hyaluronate and free hyaluronic acid, but without an isochlorin composition. At intervals of 2, 4 and 6 months, participants are asked to report level of pain relief. Participants in Group G report greater pain relief than Group H.

### Example 6—Increased Stability and Extended Shelf-Life

The following examples illustrate that CHLcu (e.g., at concentrations of 50, 100, and 250 ppm) helps stabilize and extend the shelf life of HA based on assessment of the HA viscosity profile at accelerated temperature storage conditions. The examples further demonstrate the longer-term bioactivity of HA vis-à-vis enzymatic degradation by hyaluronidase.

Viscosity measurements are made of vials of sterile, injectable grade, high molecular weight 1% sodium hyaluronate solution (Hycoat® Sterile Wound Management Solution; Hyaluronate Sodium, Solution 20 mg/2 mL; avg. molecular weight 1.0-1.3 million Daltons; supplied by Hymed Group, Bethlehem, Pa.) containing Chlorophyllin Copper Complex Sodium at varying concentrations as summarized in the table below. Preferably, dissolved and atmospheric oxygen is purged from the HA solution by displacing the headspace in the vial with Nitrogen or Argon.

| | Concentration CHLCu | | | | | | | | | | | |
|---|---|---|---|---|---|---|---|---|---|---|---|---|
| | None | | | 50 ppm | | | 100 ppm | | | 250 ppm | | |
| storage | Temp | | | | | | | | | | | |
| time | 25° C. | 40° C. | 50° C. | 25° C. | 40° C. | 50° C. | 25° C. | 40° C. | 50° C. | 25° C. | 40° C. | 50° C. |
| Initial | x | | | x | | | x | | | x | | |
| 1 week | | x | x | | x | x | | x | x | | x | x |
| 2 weeks | x | x | x | x | x | x | x | x | x | x | x | x |
| 4 weeks | x | x | x | x | x | x | x | x | x | x | x | x |
| 6 weeks | x | x | x | x | x | x | x | x | x | x | x | x |
| 8 weeks | x | x | x | x | x | x | x | x | x | x | x | x |

Sodium copper chlorophyllin complex (disodium copper isochlorin e4 minimum content of 25% based on dry weight of complex; supplied by Frontier Scientific, Logan, Utah) is added to the 1% HA test solutions (i.e., 2 mL Hycoat® described above) as follows: 1 gram of CHLcu is added to 99 grams of distilled water, heated to 50° C., mixed for 30 minutes with a magnetic stirrer, and then cooled to room temperature. The specified concentration of CHLCu solution is drawn into a syringe through a 0.45u syringe filter and added to the sterile vial of 1% HA solution under a laminar flow hood. For example, in order to prepare a 1% HA solution containing 100 ppm of CHLcu in the final HA solution 20.5 microliters (0.0205 mL) of the 1% CHLCu solution is injected into the vial of 2 mL of sterile HA solution.

Each of the 2 mL test samples (with and without CHLcu) are stored at 25° C., 40° C., and 50° C. for up to eight weeks. Viscosity measurements are taken at baseline and at 1, 2, 4, 6 and 8 weeks after storage at the three temperatures.

0.5 mL of the test sample is then withdrawn from each vial, causing a temporary shear in the sample viscosity.

All samples (1% HA+CHLCu) are equilibrated prior to viscosity measurement. More particularly, for five minutes, each vial is placed in a constant temperature water bath heated and cooled to a constant 25° C., +/−0.2° C. (e.g., Model TC-550AP-115 from Brookfield Engineering Laboratories, Inc. Middleboro, Mass.).

Each withdrawn sample is allowed to further equilibrate for five additional minutes before taking viscosity measurements. Viscosity is preferably measured at 2 rpm with a cone-plate viscometer (e.g., Brookfield LVD-II Pro Cone/Plate viscometer; CPE-51 spindle from Brookfield Engineering Laboratories, Inc. Middleboro, Mass.).

In addition to viscosity, all samples are examined for color, clarity, presence of precipitate or any other signs of instability. pH measurements are taken at baseline and after 4 and 8 weeks.

Samples to which CHLcu is added exhibit less of a viscosity change than samples not containing CHLCu.

In order to assess the anti-hyaluronidase activity of the CHLcu stabilizing agent, 40 units (0.2 mL) of Vitrase® injectable sterile hyaluronidase solution (Ovine, 200 USP Units/mL) is added to each of four 1% HA samples that have been stored at 25° C. for six weeks with (a) no CHLCu; (b) 50 ppm CHLCu; (c) 100 ppm CHLCu; and (d) 250 ppm CHLCu). Anti-hyaluronidase activity is measured by comparing the viscosity of the test samples before and after addition of Vitrase®.

A product containing 1% HA (Hycoat® Sterile Wound Management Solution; Hyaluronate Sodium, Solution 20 mg/2 mL; average molecular weight 1.0-1.3 million Daltons) and 100 mcg/mL CHLCu was combined with 40 units (0.2 mL) of Vitrase® injectable sterile hyaluronidase solution (Ovine, 200 USP Units/mL) and stored at 25° C. for 30 minutes. The viscosity of the combined solution of sodium HA, CHLCu, and Vitrase® composition was seen to drop from 450.6 cps to 161.8 cps, or 35.9% of initial viscosity.

By way of comparison, the viscosity of the same 1% HA solution without CHLCu was measured, and then exposed to 40 units of the same Vitrase. After 30 minutes of Vitrase® contact, the 1% HA solution underwent a viscosity change from 516.2 cps to 43.69 cps or only 8.5% of the initial HA viscosity.

### Example 7—Effect of Chlorophyllin Copper Complex Sodium on the Expression of Collagen Genes

Using adult human dermal fibroblasts (Cell Applications, San Diego, Calif.) (“HDF”), the effectiveness of three chlorophyllin materials—Isochlorin E4 Disodium (“E4”); Sodium Copper Chlorophyllin C3999 (“C3999”); and Chlorophyllin Sodium Copper Salt (“CSCS”)—are assessed for the ability to positively effect the differential expression of at least one of the following three collagen genes—COL15A1; COL1A1; and COL5A1. After cell viability tests, the effect of 2 μg/ml E4, 10 μg/ml CSCS, and 10 μg/ml C3999 on adult human dermal fibroblasts (100,000 cells/well) on gene expression were evaluated using PCR arrays.

HDF cells were incubated for 24 hours in a 12-well plate at a concentration of 100,000 cells/well in Dulbecco's Modified Eagle Medium (DMEM) with 5% Fetal Bovine Serum (FBS). The three test materials were added to the wells, incubated for 24 hours. Cell cultures were observed in bright field with the inverted Amscope IN300TC-FL microscope; images were captured with color Discovery 15 CMOS microscope video camera using ISCapture software. RNA was extracted and purified with NucleoSpin RNA II kit from Machery-Nagel (Bethlehem, Pa.). Purified total RNA was analyzed at 230 nm, 260 nm and 280 nm with Agilent HP-8452A diode array spectrophotometer. The concentration of RNA was equalized across the samples and the expression of genes of interest was measured by real-time quantitative PCR with BioRad iCycler iQ Detection System using Human Extracellular Matrix & Adhesion Molecules PCR array PAHS-13ZA from Qiagen (formerly SA Biosciences, Frederick, Md.). Reactions were performed with 1st strand synthesis kit, SYBR Green master mix and PCR setup parameters from Qiagen. Results were normalized to housekeeping genes with the RT2 Profiler PCR Array Data Analysis version 3.5 software

Genes were considered differentially expressed if the level of expression was detected in less than 30 cycles, and modulation was 1.5 fold or higher.

| | CSCS 10 μg/ml | C3999 10 μg/ml | E4 2 μg/ml |
|---|---|---|---|
| COL15A1 | 1.2 | 1.5 | 1.2 |
| COL1A1 | 1.5 | 1.2 | 1.2 |
| COL5A1 | 1.7 | 1.3 | 1.5 |

### Example 8—Long Term HDF Viability with E4

The effect of long-term incubation of HDF with E4 is assessed by MTT, a colorimetric assay for assessing cell metabolic activity. Cells were found to be unaffected by 1 μg/mL.

### Example 9—Effect of Chlorophyllin Copper Complex Sodium on the Collagen Production by HDFs

The same test materials from Example 10—E4, CSCS, C3999—is further evaluated for their effect on the production of soluble and non-soluble Type I collagen by adult human dermal fibroblasts.

Adult human dermal fibroblasts (Cell Applications, San Diego, Calif.) were plated in DMEM without phenyl red supplemented with 5% FBS and 1% penicillin/streptomycin (PS) at a concentration of 8,000 cells/well. Test materials, which were stored at 4° C. until dissolved at 20 mg/ml in ddH2O, were added the next day on attached exponentially-growing cells. Negative and positive controls were, respectively, type I sterile water and L-Ascorbic acid 2-phosphate sesquimagnesium salt (“MAP”) (Sigma-Aldrich).

After 4 days of incubation with test materials, cell culture conditioned media were harvested and tested for soluble Type I collagen by sandwich ELISA using anti-Type I collagen affinity-purified antibodies, followed by straptavidin-avidin-HRP conjugate and ABTS (SouthernBiotech, Birmingham, Ala.), according to ELISA protocols used in dermatological research—Dobak et al., “1,25-Dihydroxyvitamin D3 increases collagen production in dermal fibroblasts,” J. Dermatol. Sci., Vol. 8, pp. 18-24 (1994); and Zhao et al. “Lycium barbarum glycoconjugates: effect on whole skin and cultured dermal fibroblasts” Phytomedicine, Vol. 12, pp. 132-138 (2005).

Cells were then fixed with 2.5% trichloroacetic acid (TCA) at 4° C., dried overnight, and tested for non-soluble type I collagen, using direct ELISA.

Cells proliferation was quantified by staining cytoskeletons with sulforhodamine B according to the colorimetric method of Voigt, “Sulforhodamine B assay and chemosensitivity,” Methods Mol. Med. Vol. 110, pp. 39-48 (2005). Statistical significance was assessed with two-tailed paired Student test. Deviations of >15% as compared to the negative water control with p values below 0.05 were considered statistically significant.

The effect of test materials on non-soluble collagen I production, cell numbers, and non-soluble collagen I standardized to cell numbers expressed as % of the negative control (water) are presented in the following table. (The positive control (MAP) provided significant stimulatory impulse for both soluble and insoluble type I collagen forms, validating the experimental design.)

| | | | % CTRL | p value |
|---|---|---|---|---|
| | | | (Cell # | (Cell # |
| | % CTRL | % CTRL | Standard- | Standard- |
| Test Material | (COL-I | (Cell #) | ized) | ized) |
| H₂O | 100 | 100 | 100 | 1.000 |
| E4 10 μg/mL | 90 | 80 | 111 | 0.415 |
| E4 5 μg/mL | 94 | 81 | 115 | 0.320 |
| E4 2 μg/mL | 120 | 89 | 133 | 0.037 |
| C3999 20 μg/mL | 121 | 83 | 145 | 0.011 |
| C3999 10 μg/mL | 110 | 92 | 118 | 0.391 |
| C3999 5 μg/mL | 97 | 95 | 101 | 0.791 |
| CSCS 20 μg/mL | 84 | 88 | 94 | 0.586 |
| CSCS 10 μg/mL | 85 | 87 | 96 | 0.751 |
| CSCS 5 μg /mL | 95 | 95 | 99 | 0.736 |
| MAP 100 μg/mL | 141 | 100 | 138 | 0.004 |
| MAP 50 μg /mL | 132 | 111 | 117 | 0.171 |

The same testing was repeated with E4 and CSCS at different concentrations.

Additionally, the stimulatory effect of the combination of E4 and CSCS on type I collagen deposition by human dermal fibroblasts was also evaluated. At the tested concentrations, E4/CSCS combination had a slightly higher overall stimulatory profile than CSCS and E4, individually.

| Test Material | Cell # Standardized |
|---|---|
| H₂O | 100 |
| CSCS 5 μg/ml | 110 |
| CSCS 2 μg/ml | 114 |
| CSCS 1 μg/ml | 96 |
| CSCS 0.25 ug/ml | 97 |
| E4 5 μg/ml | 101 |
| E4 2 μg/ml | 113 |
| E4 1 μg/ml | 89 |
| E4 0.25 ug/ml | 88 |
| CSCS:E4 (1:1) 5 μg/ml | 116 |
| CSCS:E4 (1:1) 2 μg/ml | 120 |
| CSCS:E4 (1:1) 1 μg/ml | 114 |
| CSCS:E4 (1:1) 0.25 ug/ml | 104 |

### Example 10—Improvement in Photoaged Skin as Measured by Biomarkers in Human Extracellular Matrix

A study is conducted to determine the effect of CHLCu on the expression of biomarkers of photoaged dermal extracellular matrix indicative of skin repair.

Four healthy women with signs of photoaged skin participated in a twelve-day study according to the experimental design used by Watson et al and reported in the Br J Dermatol. 2008; 158(3):472-477. Each participant was treated with a composition of the present invention (a gel comprised of 0.05% of CHLCu in a liposomal dispersion), a positive control of Tretinoin Cream, 0.025% and an untreated negative control. Three 4×5 cm patch sites were marked on the extensor surface of the left or right forearm of each participant according to a predetermined randomization. On day 1, 4, 6, 8, and 10, 50 μL of the composition of the present invention was applied topically to one of the randomized marked patch sites. On day 8, 50 μL of the reference control material (Tretinoin 0.025% Cream) was applied to an assigned patch site. In order to minimize the potential for skin irritation, the control was allowed to remain on the participants' skin for 4 days. The remaining site was untreated during the course of the study to serve as a negative control. All 3 test sites were covered with Finn Chambers (Allerderm Laboratories, Inc., Mill Valley, Calif., USA) 12 mm inner diameter aluminum chambers affixed to Scanpore Tape (Norgesplaster A/S, Norway) on each application day.

Clinical evaluations were conducted at visit 2 (day 4), visit 3 (day 6), visit 4 (day 8), visit 5 (day 10), and visit 6 (day 12) for grading reaction (i.e., erythema) and scoring of test sites.

Since it was not possible to directly measure HA content in the biopsy specimens, assessments of changes in epidermal and dermal mucins were used as a surrogate for measuring changes in hyaluronan.

3-mm punch biopsies collected from each of the 3 test sites at day 12. After collection, biopsy samples were transferred into 10% neutral buffered formalin solution and stored at room temperature overnight. Samples were shipped to a laboratory the next day for processing by paraffin embedding. Tissue sections were then processed for immunohistochemistry staining for Pro-collagen 1, Fibrillin 1 surrogate stain Amyloid P, and mucins (dermal and epidermal; by both colloidal iron and Alcian blue staining methods). Expression of these markers was graded by an expert grader using the following scales; half-point scores were acceptable.

Procollagen 1: Sample slides were processed for immunohistochemistry staining for Pro-collagen Type 1 using MAB1912 Anti-Procollagen Type I Antibody, N-terminus, clone M-58 from EMD Millipore (Billerica, Mass.), according to the manufacturer's directions. Antibody dilutions of 1:100 to 1:1000 were used, after treatment with 1% trypsin, 20 minutes at room temperature. Staining was scored on the following scale, in which % refers to percentage of cells with positive staining: 1=<5% cells; 2=5-10% cells; 3=10-20% cell; 4=20-30% cells; 5=>30% cells.

Fibrillin 1: Sample slides were processed for immunohistochemistry staining for Fibrillin 1 surrogate stain Amyloid P using Anti-Serum Amyloid P antibody [EP1018Y] (ab45151), a rabbit monoclonal antibody to Serum Amyloid P, from Abcam (Cambridge, Mass.) according to the manufacturer's directions. Antibody dilutions of 1:100 to 1:250 were used. Heat mediated antigen retrieval with citrate buffer pH6 was performed before commencing with IHC staining protocol. Staining was scored on the following scale: 0=within normal limits; 1=mild increase in dermal reticulin fibers; 2=moderate increase in dermal reticulin fibers; 3=marked, diffuse increase in dermal reticulin fibers.

Mucin: Two different histology methods were employed for epidermal and dermal mucin staining, colloidal iron staining and Alcian Blue staining before and after digestion with hyaluronidase; the latter method was used to confirm the results of the colloidal iron staining method after the removal of HA-related proteoglycans with hyaluronidase.

Colloidal Iron Staining: Skin biopsy samples (fixed in 10% formalin for 20-24 hours prior to embedding in paraffin) was cut at 4 microns onto positively charged slides and the sections deparaffinized and rehydrated in distilled water. Slides were rinsed briefly in 12% acetic acid solution and placed in working colloidal iron solution for one hour (Muller's colloidal iron solution+glacial acetic acid+DI water). Slides were then immersed in Ferrocyanide-Hydrochloric acid solution for 20 minutes at room temperature, after which the slides were rinsed in tap water. The slides were then counterstained with nuclear fast red solution for 5 minutes. Slides were rinsed again in tap water, dehydrated in 95% alcohol and absolute alcohol, cleared in xylene (×2), and coverslipped. Slides were visualized via conventional light microscopy for mucin deposition within the epidermis and dermis (stringy-blue hue), and graded on the following semi quantitative scale: 0=normal; 1=mild increase in interstitial mucin; 2=moderate increase in interstitial mucin; 3=marked mucin deposition.

Alcian Blue Staining Before and After Digestion with Hyaluronidase: Skin biopsy samples were fixed in 10% formalin for 20-24 hours prior to embedding in paraffin and cut as above (at 4 microns onto positively charged slides and the sections deparaffinized and rehydrated in distilled water). Slides were placed in 3% acetic acid solution for 3 minutes, and then in Alcian blue solution (pH 2.5, 1% Alcian blue 8GX, 3% acetic acid, thymol) for 30 minutes. Following a tap water rinse, slides were incubated in 0.5% periodic acid for 10 minutes, rinsed, and then placed in Schiff's reagent for 10 minutes. Slides were then rinsed again in tap water, dehydrated in 95% alcohol and absolute alcohol, cleared in xylene (×2), and coverslipped. Slides were visualized via conventional light microscopy for mucin deposition within the epidermis and dermis (stringy-blue hue), and graded on the same semi quantitative scale as was used for colloidal iron staining.

For hyaluronidase treatment, deparaffinized slides (4 microns) were treated with hyaluronidase solution (0.05 g lyophilized hyaluronidase in 100 mL of 0.1 M hyaluronidase buffer) for 1 hour at 37° C. Following a tap water rinse, slides were stained per Alcian blue protocol and visualized/graded accordingly.

The difference between histology scores (epidermal and dermal mucin are rated on four point (0-3) scale: 0=normal, 3=marked increased staining) with Alcian blue stain before digestion and after digestion with hyaluronidase was the final score for analysis. All post digestion scores with Alcian blue were zero and scores for test treatment, positive control and untreated control were identical to the colloidal iron histology results indicating that the mucin changes were due to hyaluronan.

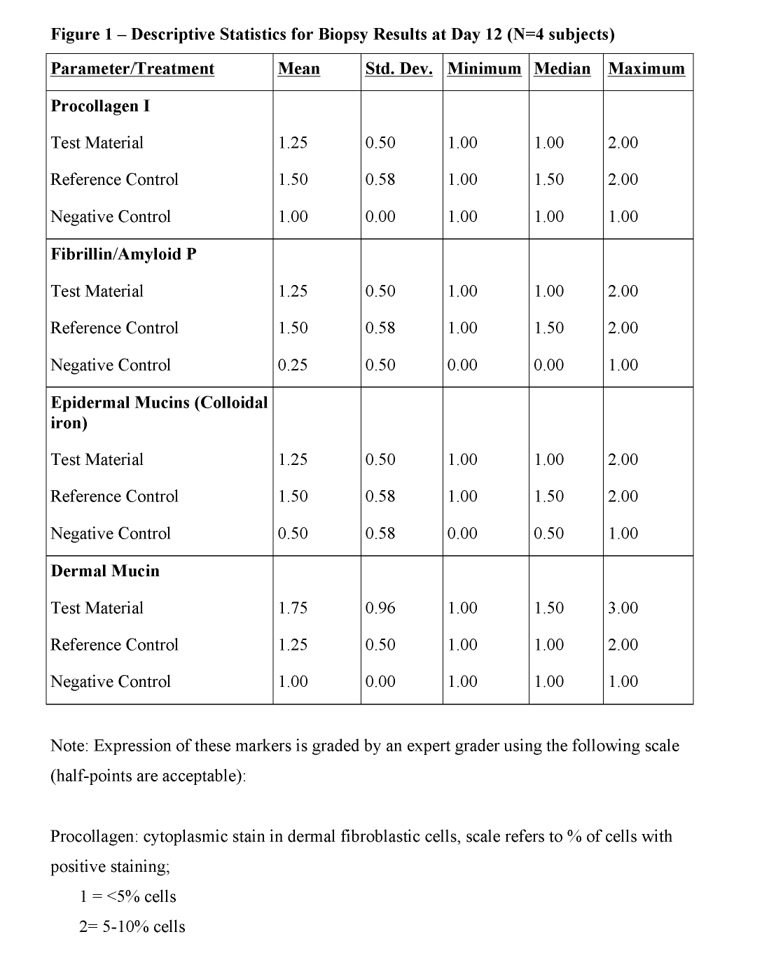

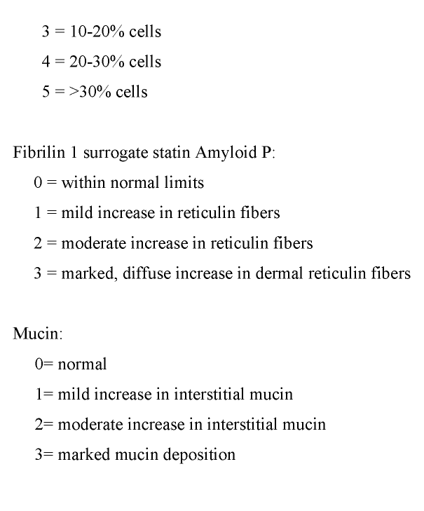

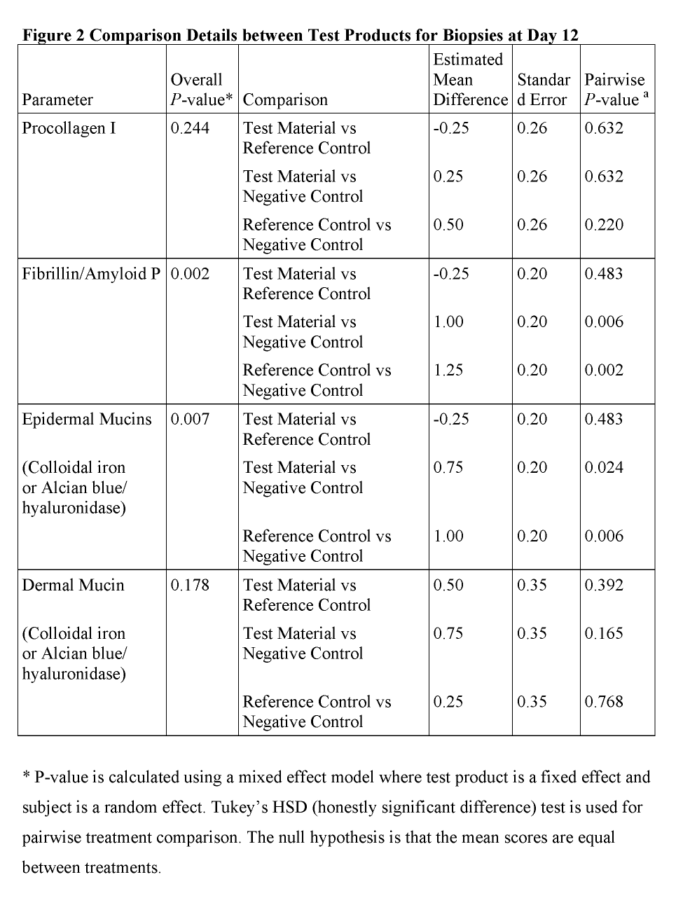

Biopsy analyses are presented in table form as FIGS. 1 and 2. Both the composition of the present invention and the positive control, Tretinoin Cream 0.025%, increased the presence of Procollagen 1, Fibrillin 1, and epidermal and dermal mucins, biomarkers of dermal repair, compared to the negative control. The changes in Fibrillin 1 and epidermal mucins for both the composition of the present invention and the positive control were statistically significant (p<0.05) when compared to no treatment.

1. (canceled)

2. (canceled)

3. (canceled)

4. The method of claim 11 wherein the non-native hyaluronic acid is modified by crosslinking with a cross-linking agent selected from the group consisting of 1,4-butanediol diglycidyl ether, di-vinyl sulfone, 1,4-bis(2,3-epoxypropoxy)butane, 1,4-bisglycidyloxybutane, 1,2-bis(2,3-epoxypropoxy) ethylene, 1-(2,3-epoxypropyl)-2,3-epoxycyclohexane, pentaerythritol tetraglicidyl, and sodium glucuronate-N-acetylglucosamine.

5. A method of extending the duration of skin plumping from an injection of a dermal filler preparation comprised of hyaluronic acid, or non-native hyaluronic acid that is modified by crosslinking with a cross-linking agent selected from the group consisting of 1,4-butanediol diglycidyl ether, di-vinyl sulfone, 1,4-bis(2,3-epoxypropoxy)butane, 1,4-bisglycidyloxybutane, 1,2-bis(2,3-epoxypropoxy) ethylene, 1-(2,3-epoxypropyl)-2,3-epoxycyclohexane, pentaerythritol tetraglicidyl, and sodium glucuronate-N-acetylglucosamine, comprising the step of (a) adding to the dermal filler preparation sodium copper isochlorin e4 or oxidized sodium copper isochlorin e4 or (b) topically administering at the injection site and tissue surrounding the injection site a composition comprising sodium copper isochlorin e4 or oxidized sodium copper isochlorin e4.

6. (canceled)

7. (canceled)

8. A method of increasing the expression of one or more genes associated with the production of one or more of collagens, fibrillins, and mucopolysaccharides by topical administration or injection of sodium copper isochlorin e4 or oxidized sodium copper isochlorin e4.

9. (canceled)

10. A method of extending the storage shelf life of a preparation comprised of hyaluronic acid or hyaluronic acid that is modified by crosslinking with a cross-linking agent selected from the group consisting of 1,4-butanediol diglycidyl ether, di-vinyl sulfone, 1,4-bis(2,3-epoxypropoxy)butane, 1,4-bisglycidyloxybutane, 1,2-bis(2,3-epoxypropoxy) ethylene, 1-(2,3-epoxypropyl)-2,3-epoxycyclohexane, pentaerythritol tetraglicidyl, and sodium glucuronate-N-acetylglucosamine, by adding to the preparation sodium copper isochlorin e4 or oxidized sodium copper isochlorin e4.

11. A method of increasing the residence time or half-life of (i) endogenous hyaluronic acid or (ii) non-native hyaluronic acid by administering, via injection or applied topically, sodium copper isochlorin e4 or oxidized sodium copper isochlorin e4 to (a) skin exhibiting rhytids, grooves, furrows, creping, sagging, or otherwise appearing hollow or (b) knees, ankles, shoulders, elbows, wrists, distal phalanges, and spine, including facet joints and intervertebral discs thereof.

12. The method of claim 11 wherein non-native hyaluronic acid is injected or applied topically and sodium copper isochlorin e4 or oxidized sodium copper isochlorin e4 is injected or applied topically.

13. (canceled)

14. The method of any of claim 11, wherein sodium copper isochlorin e4 or oxidized sodium copper isochlorin e4, is administered via injection at a concentration of from about 1 mcg/mL to about 1000 mcg/mL.

15. (canceled)

16. (canceled)

17. (canceled)

18. (canceled)

19. The method of claim 8 wherein an increase in the gene expression of one or more of COL1A1, COL5A1, and COL15A1 is caused by topical administration or injection of sodium copper isochlorin e4 or oxidized sodium copper isochlorin e4.
